(19) United States
(12) Patent Application Publication
(10) Pub. No.: US 2008/0021422 A1
(43) Pub. Date: Jan. 24, 2008

(54) Packaging Device for Topical Application of an Active Ingredient

(76) Inventors: Florence Guillet Couasnon, Saint Orens (FR); Vincent Cazelles, Ambres (FR)

Correspondence Address: BIRCH STEWART KOLASCH & BIRCH, PO BOX 747, FALLS CHURCH, VA, 22040-0747, US

(21) Appl. No.: 11/663,972

(22) PCT Filed: Sep. 27, 2005
(86) PCT No.: PCT/EP05/54857

§ 371 Date: Mar. 28, 2007

(30) Foreign Application Priority Data
Sep. 29, 2004 (FR) 0410301

(51) Int. Cl. A61M 35/00; A61F 5/41

(52) U.S. Cl. 604/311

(57) ABSTRACT

The invention relates to the use of a packing device for topically applying a formulation comprises an active vasodilating principle for treating erectile dysfunction and the device comprises a body (1) which is provided with an open base (2) for receiving at least one fingertip and an application head (3) having at least one opening. Said body also comprising a movable means (5) which defines a cavity (6) filled with the formulation (7) and makes it possible to discharge said formulation through the opening (4) by exposing said mobile means to a pressure.

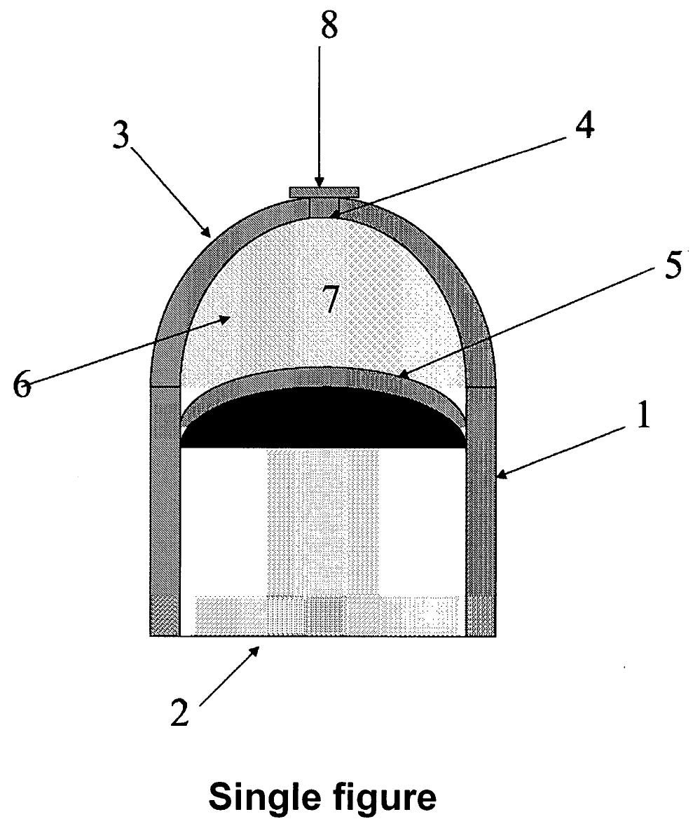

The present invention relates to a device for the application of a formulation of a pharmaceutical active ingredient on the mucosa, and more particularly, the application of a vasodilator active ingredient on the penile mucosa in order to treat erectile dysfunction.

Erectile dysfunction, or impotence, is characterized by the incapacity to obtain and/or maintain an erection sufficient to enable satisfactory sexual relations.

The causes of impotence can be multiple and of physiological or psychological origin.

Among these causes, on the one hand, lesions of the central nervous system or of the perineal area can be cited. On the other hand, vascular problems, neurodegenerative disorders or traumas can be the cause of temporary or permanent impotence.

In the field of erectile dysfunction treatment, various therapeutic solutions exist.

One possible solution consists of internal prostheses; such implants, however, require a surgical procedure, are expensive and are not always satisfactory.

The primary treatment solution for erectile dysfunction is the administration of a drug. Knowing that an erection involves vasodilatation of the penile arteries, the physiological causes of impotence are often treated using vasodilator active ingredients.

A specific problem lies in the route of administration of the vasodilator active ingredient. Indeed, it is essential that the vasodilatation effect is achieved as soon as possible after administration of the active ingredient, with the shortest possible latency time. Thus, systems of intraurethral administration or intracavernous injection of vasodilator active ingredients have been developed in order to treat erectile dysfunction. However, intraurethral administration involves the use of a medical device that is often intricate and uncomfortable for the patient. With respect to intracavernous injection, it goes without saying that such a mode of administration is particularly delicate and is not generally tolerated well by patients. Such problems related to application comfort have led to the development of formulations for topical transmucosal application directly on the penis and/or the foreskin in the form of creams, gels or ointments.

The topical transmucosal mode of administration is well suited for the application concerned and various products can be found on the market formulated of a vasodilator combined with one or more penetration agents or absorption facilitators which serve to facilitate and accelerate migration of the vasodilator active ingredient into the cavernous bodies in which the vasodilator carries out its physiological role.

Among the vasodilator active ingredients commonly used for erectile dysfunction, the prostaglandins, particularly prostaglandin E1 (PGE1) or prostaglandin E2 (PGE2), can be cited as examples.

The application of topical transmucosal formulations to treat erectile dysfunction is carried out by the patient by means of manually spreading a suitable quantity of the product from a tube containing the preparation. Such a manual application can pose certain problems. First, repeated contact between the hands or fingers and the product is not always desirable from a sanitary point of view because such contact can cause contamination of the product. Another significant problem is that the manual application of vasodilators leaves a residual quantity of the product on the skin of the fingers. Thus, unless gloves are worn or the hands are washed quickly after application, the presence of the vasodilator on the hands can lead to the local inflow of blood. Moreover, knowing that topical transmucosal formulations include one or more agents that support transfer of the active ingredient, absorption of said active ingredient is rapid, including in the hands. The inflow of blood thus generated in the hands and fingers involves discomfort, even pain, for the user during the application of such products. For topical application on the penile mucosa, a device exists of the “wafer” type, i.e., using a very thin, rapidly-disintegrating film that is applied to the glans. Said film is comprised of a vasodilator active ingredient which is absorbed once said film is applied to the penile mucosa. However, the manufacture of such a “wafer” is complex.

The packaging and application systems intended for creams, gels, pomades and ointments currently described are generally in the form of a container, optionally under pressure, with which, also optionally, is combined a generally tubular applicator whose function is to direct the product towards the area to be treated. This kind of packaging and application device does not respond optimally to the specificity of the topical application of a vasodilator for which a certain period of massaging is useful to ensure the satisfactory absorption of the active ingredient, and this is true in spite of the possible presence of a penetration agent. In the case of application on the mucosa, the aforesaid devices described in the prior art do not permit application by direct contact for obvious reasons of hygiene.

Consequently, there is a need for a device intended for and adapted to the packaging and extemporaneous topical application of an active ingredient on the mucosa, in particular of a vasodilator active ingredient, more particular still on the penile mucosa.

To this end, the present invention relates to a packaging device for the topical application of a formulation comprised of an active ingredient on the mucosa, the aforesaid packaging system comprising: a body (1) having an open base (2) for receiving at least one fingertip and an application head (3) having at least one opening (4), said body also comprising a moveable means (5) which defines a cavity (6) filled with the formulation (7) and which allows the discharge of the aforesaid formulation through the opening (4) when pressure is exerted on the moveable means.

The use of such a device has a number of advantages. There is no contact between the active ingredient and the skin of the user except where the application is desired, on the mucosa; thus, there are no side effects by the active ingredient at unintended locations. In a specific mode of use, the implementation of such a device is adapted to the application of the aforesaid active ingredient by someone other than the patient.

The single FIGURE presents a longitudinal cross-section of a specific embodiment of the device according to the present invention.

The open base (2) located on the body (1) has the dimensions and shape suitable for placing one or more fingertips within the applicator, which facilitates topical application of the active ingredient. Thus, the open base (2) will preferentially have an annular cross-section suitable for the finger of a user. The diameter of the opening is not critical and it may be selected according to the public concerned.

The application head (3) is provided with at least one sealable opening (4) for dispensing the formulation (7) comprising the active ingredient contained in the cavity (6) of the aforesaid device. The size and shape of the opening (4) are not critical and they may be selected, for example, according to the viscosity of the aforesaid formulation comprising the active ingredient. Said opening (4) may be a single opening or multiple openings of circular or ovoid shape, for example. The sealing of this opening can be carried out via any sealing means (8) known to those skilled in the art, keeping in mind that no residue should remain projecting from the application head in order to avoid any risk of injury during application. Indeed, the use according to the present invention is intended for application on the mucosa, tissue that is particularly sensitive and fragile. Thus, a self-adhesive, threaded or welded cap sealing the opening can be considered as the means of sealing (8), a cap which is withdrawn when the applicator is used. In the case of a single opening, a cap plug, which may be held in position by threads or clips, may be considered.

The application head (3) will preferably be of hemispherical shape to ensure precision and comfort during application. However, a flat application head, a slightly convex application head or a combination thereof can also be considered. In particular, a truncated hemispherical shape is preferred because said shape permits better sealing of the opening. It must be kept in mind, however, that shapes with angles that project too much are likely to injure the user or patient during application and thus must be avoided.

In a general way, the device according to the present invention will not have sharp angles or projecting residues with respect to the areas intended to come in contact with the mucosa during application. This makes it possible to avoid any injuries, knowing indeed that the use according to the present invention is intended for the mucosa. The application according to the present invention relates to healthy or injured mucosa, preferably healthy.

The moveable means (5), present in the body of the device and delimiting the cavity (6) containing the formulation (7) comprising the active ingredient, acts as piston to expel the active ingredient during use. The user places one or more fingers in the open base of the body and, by exerting pressure along the longitudinal axis, expels the formulation containing the active ingredient through the opening once the seal is removed from said opening.

The moveable means (5) will be designed to ensure the best possible seal at its junction with the body of the device so as to avoid any leak of the formulation containing the active ingredient. This moveable means may be an annular piston optionally equipped with an extendible annular membrane. This piston may also be fitted with two lips that slide along the wall of the body. In the case of a piston, a convex piston can be considered, one which is adapted to the shape of the application head and which thus allows the most complete possible expulsion of the formulation contained in the cavity once the aforesaid piston arrives at the stop position. A membrane or deformable wall, defining a cavity filled with the formulation containing the active ingredient and allowing the expulsion of the active ingredient through the opening once pressure is exerted on the aforesaid membrane or deformable wall which thus becomes deformed, may also be considered. The material used for the aforesaid piston will be compatible with the pharmaceutical active ingredients that comprise the formulation contained within the cavity. Thus, said material may be selected from the group of polyolefins or of elastomers, for example. In particular, polyethylene (PE) is particularly suitable, more preferentially high-density polyethylene (HDPE).

It can also be seen that the use of such a device is particularly suitable for the application of an active ingredient on the mucosa. In particular, during the application of a vasodilator active ingredient for the purpose of treating erectile dysfunction, the use of such a device makes it possible to avoid the disadvantages previously mentioned. Thus, the application can be accomplished without the active ingredient coming in contact with any skin other than that of the penile mucosa for which such an active ingredient is intended.

The present invention also relates to a method for treating erectile dysfunction in which an active ingredient contained in a pharmaceutical formulation intended for the topical treatment of the aforesaid erectile dysfunction is applied to the penile mucosa using a device comprising: a body (1) having an open base (2) for receiving at least one fingertip and an application head (3) having at least one opening (4), said body also comprising a moveable means (5) which defines a cavity (6) filled with the formulation (7) and which allows the discharge of the aforesaid formulation through the opening (4) when pressure is exerted on the moveable means.

Within the framework of the present invention, “penile mucosa” means the surface of the glans. This may concern the external mucosa as well as the internal mucosa, namely the urethral meatus. The use according to the present invention can also be considered to topically dispense an active ingredient on any other mucosa for which direct contact with the hand or fingers is not desirable or practical. Thus, the oral, anal or vaginal mucosa can also be concerned by a use according to the present invention. With respect to the vaginal mucosa, the use of such a device lends itself particularly well to the local application of antibiotics or antimycotics. The use of the aforesaid device according to the invention can also be considered for the topical oral application of an active ingredient, for the treatment of mouth ulcers and/or gingivitis, for example.

The active ingredient concerned by the use according to the present invention can be any active ingredient that can be at least potentially applied topically on the mucosa. Preferentially, the active ingredient is a vasodilator. Among the suitable vasodilator active ingredients, alpha-1 blockers and/or alpha-2 blockers such as phentolamine or moxisylyte and PDE-5 (phosphodiesterase-5) inhibiters can be cited. More preferentially still, the active ingredient comprises at least one prostaglandin, preferably the prostaglandin E1 alprostadil. In the use of the aforesaid device for erectile dysfunction, any active ingredient with vasodilating action by topical route can be considered within the framework of the present invention. Thus, the vasodilator active ingredient may be selected from the group comprising alprostadil, misoprostol, papaverine and phentolamine. The vasodilator active ingredient may also be misoprostol as described in patent EP 980245, for example. As indicated above, in the case of application on vaginal mucosa, the active ingredient delivered may be selected from the group comprising antibiotics and antimycotics.

The vasodilator active ingredient applied in the use according to the present invention can be formulated according to standard compositions known to those skilled in the art. A suitable formulation can be a formulation in the form of a gel or cream, whose viscosity can reach 45,000 centipoises at an ambient temperature of approximately 20° C., containing an effective quantity of the vasodilator active ingredient. The use according to the present invention is suitable for any formulation of a vasodilator active ingredient from the point at which said formulation ensures adequate viscosity for expulsion of the composition containing the active ingredient and for topical application on the mucosa. An adequate formulation containing a vasodilator active ingredient such as misoprostol is described in the patent EP 980245.

The volume of the formulation comprising the active ingredient contained in the cavity defined by the body and the moveable means will be selected according to the indication concerned. In the case of treatment of erectile dysfunction, the quantity of product may be approximately 0.1 ml to 3 ml, preferentially from approximately 0.2 ml to 2 ml, and more preferentially still from approximately 0.5 ml to 1 ml. This quantity will, however, be limited by the size of the packaging system, itself adapted to the size of one or more fingers because said system is configured for such a use as a finger-operated device.

For the indication of erectile dysfunction, the volume of the formulation to be delivered topically will be selected so as to ensure comfort and convenience of use for the patient while ensuring the delivery of an effective quantity of vasodilator active ingredient to ensure a sufficient erection. In this case, the surface of the mucosa to be treated is relatively restricted and too great a volume of the formulation comprising the vasodilator active ingredient would be inadequate because a quantity of the formulation, and therefore of the active ingredient, would likely be lost.

In an advantageous way, the body of the packaging and application system according to the present invention may be tinted using an agent making said system opaque, thus avoiding any risk of degradation of the active ingredient by ultraviolet rays. The body of said system will be made of a rigid material such as a polymer chosen from the group comprising polyolefins, polyesters and styrene compounds. Advantageously, said body may be made of polypropylene.

Within the framework of a use for the indication of erectile dysfunction, the packaging and application system, as well as the amount contained therein, will be sized for unit application. Such a single dose unites the advantages inherent and necessary for this indication. Thus, the active ingredient is protected from external degradation agents and extemporaneous opening ensures optimal hygiene for the patient.

The packaging and application systems according to the present invention may be packaged individually in blister packaging or in bags, optionally aluminized, under a protective atmosphere, for example.

1. A use of a packaging device for the topical application on the mucosa of a formulation comprising a vasodilator active ingredient for the treatment of erectile dysfunction, wherein the device comprises a body (1) having an open base (2) for receiving at least one fingertip and an application head (3) having at least one opening (4), said body also comprising a moveable means (5) which defines a cavity (6) filled with the formulation (7) and which allows the discharge of the aforesaid formulation through the opening (4) when pressure is exerted on the moveable means.

2. A use according to claim 1, wherein the open base has an annular cross-section.

3. A use according to claim 1, wherein the application head has a convex, preferably hemispherical, shape.

4. A use according to claim 1, wherein the moveable means is a piston.

5. A use according to one of the claims 1 to 4, wherein the opening is comprised of multiple openings.

6. A use according to one of the claims 1 to 5, wherein the opening is sealed using a means of sealing (8) such as a cap that is self-adhesive, threaded or held by clips.
